(19) United States
(12) Patent Application Publication
(10) Pub. No.: US 2005/0096514 A1
(43) Pub. Date: May 5, 2005

(54) Gastric activity notification

(75) Inventor: Warren L. Starkebaum, Plymouth, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 10/698,115

(22) Filed: Nov. 1, 2003

(51) Int. Cl. A61B 5/00; A61B 5/04; A61B 5/05; A61B 5/103

(52) U.S. Cl. 600/309; 600/365; 600/547; 600/549; 600/587; 600/546; 128/903

(57) ABSTRACT

In general, the invention is directed to methods and devices for monitoring one or more physiological parameters that reflect the activity of the stomach of a patient, and generating a communication notifying the patient as a function of the sensed parameters. Upon receiving notification, the patient may modify his activity by discontinuing eating, for example, or by self-administering medication.

### FIELD OF THE INVENTION

The invention relates to medical devices and methods, and in particular, to medical devices and methods that monitor physiological activity of the stomach.

### BACKGROUND

In a number of circumstances, it is desirable to monitor physiologic activity of the stomach of a patient. Stomach activity may be useful to help the patient control his obesity, manage his diabetes, monitor his gastroesophageal reflux disease (GERD), and the like. As part of his control over his own health, a patient monitors the kinds or amounts of foods he eats. In some cases, however, the patient could benefit from additional information about his gastric activity. In particular, the patient could benefit from feedback that he could use to modify his eating behavior, for example, or to regulate administration of his medication.

In general, monitoring of the activity of the stomach of the patient has been largely left up to the patient. In some circumstances, an implanted device monitors gastric activity, but does not support the patient's own efforts to regulate his behavior. Table 1 below lists examples of documents that disclose techniques for monitoring gastric activity.

TABLE 1

| Patent Number | Inventors | Title |
|---|---|---|
| 20020072780 | Foley | Method and apparatus for intentional |
| | | impairment of gastric motility and/or |
| | | efficiency by triggered electrical |
| | | stimulation of the gastrointestinal tract |
| | | with respect to the intrinsic gastric |
| | | electrical activity |
| 6,327,503 | Familoni | Method and apparatus for sensing and |
| | | stimulating gastrointestinal tract |
| | | on-demand |
| 5,938,669 | Klaiber et al. | Adjustable gastric banding device for |
| | | contracting a patient's stomach |
| 5,341,803 | Goldberg et al. | Apparatus and method for monitoring |
| | | gastric fluid pH |

All documents listed in Table 1 above are hereby incorporated by reference herein in their respective entireties. As those of ordinary skill in the art will appreciate readily upon reading the Summary of the Invention, Detailed Description of the Preferred Embodiments and Claims set forth below, many of the devices and methods disclosed in the patents of Table 1 may be modified advantageously by using the techniques of the present invention.

Monitoring physiologic activity manually has significant drawbacks. The patient must adhere to a strict regimen to periodically monitor activity, often several times a day, and constantly be aware of symptoms that indicate additional treatment.

### SUMMARY

The present invention has certain objects. That is, various embodiments of the present invention provide solutions to one or more problems existing in the prior art with respect to prior techniques for monitoring gastric activity. These problems include the lack of feedback to the patient about his stomach activity. Natural feedback mechanisms, such as the normal sensation of fullness following a meal, may be insufficient for a patient to regulate his own behavior. An obese patient, for example, may continue to consume food after being full because of a delay between onset of fullness and the onset of the sensation of fullness. An obese patient may benefit from information about fullness that precedes the natural sensation. An obese patient may also benefit from knowing the size of a meal, which is related to caloric intake.

Similarly, a diabetic patient may benefit from knowing the size of a meal, because the size of the meal is related to blood glucose concentrations. With this knowledge, the patient may regulate administration of his medication. Feedback about stomach activity may also aid a patient suffering from GERD, who may use the information to adjust his food intake. Knowledge about stomach activity may be useful to other patients as well.

The present invention has the object of solving at least one of the foregoing problems. For example, it is one object of the invention to monitor one or more physiological parameters that vary as a function of stomach activity. Distension of the stomach is one example of one physiological parameter reflecting activity of the stomach that may be monitored by the invention. When embodied as an implantable device, the invention includes sensor to sense the physiological parameter. The invention also includes a processor that generates a communication to the patient as a function of the sensed physiological parameter. The patient may be notified by an external module or by an implanted alert module.

The processor monitors one or more physiological parameters and may measure various characteristics of a physiological parameter, such as a rate of change, an amplitude, a duration, an intensity and a concentration. The processor can evaluate whether a characteristic should be brought to the attention of the patient, and may generate a communication as a function of the measured characteristic. Extreme distension of the stomach of a particular patient, for example, may result in generation of a communication, while mild distention will not result in generation of a communication to that patient.

The invention provides considerable freedom and enjoyment of life for the patient. In various embodiments, the patient can use the invention to obtain information about his condition, and to exercise control over his own health and well-being.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a diagram illustrating devices for monitoring activity of the stomach and notifying the patient of stomach activity.

FIG. 2 is a block diagram illustrating constituent components of an embodiment of the invention depicted in FIG. 1.

FIG. 3 illustrates a graphical representation of an exemplary sensed physiological parameter over a period of time.

FIG. 4 is a flow diagram illustrating a technique for generating a communication as a function of a sensed physiological parameter.

### DETAILED DESCRIPTION

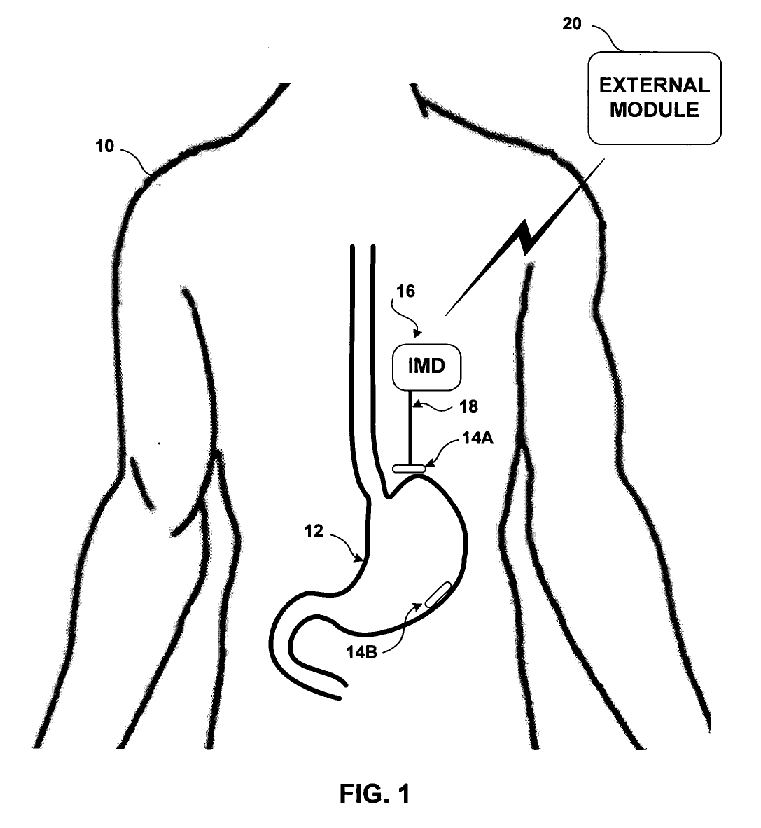

FIG. 1 is a block diagram illustrating a view of a torso of a patient 10, in which stomach 12 is visible. FIG. 1 illustrates devices for monitoring physiologic activity of the stomach 12 and notifying patient 10 as a function of the monitored activity.

Parameters such as blood glucose or insulin concentration, core body temperature, distention of the stomach, and pH level of the stomach may have a bearing upon the health of patient 10. Each of these parameters varies as a function of food intake. As a result, stomach activity may pertinent various health-related parameters. In addition, stomach activity may be of interest when patient 10 is trying to lose weight.

In FIG. 1, sensors 14A and 14B (hereinafter generally 14) sense physiologic activity of stomach 12. Sensor 14A is implanted in the body of patient 10, but is external to stomach 12. Sensor 14A is coupled to an implantable medical device (IMD) 16 by a lead 18. Sensor 14B, by contrast, is deployed inside stomach 12, and may communicate with IMD 16 wirelessly. The invention is not limited to deployment of two sensors, nor is the invention limited to deployment of sensors at the sites shown in FIG. 1.

Sensor 14 may be any sensor that senses or responds to any physiological parameter that reflects activity of stomach 12. In some embodiments, sensor 14 includes one or more electrodes to detect gastric electrical activity, transabdominal impedance, or other electrical indicators of stomach activity. In other embodiments, sensor 14 includes a chemical sensor that detects blood glucose, stomach acid, or other chemical indicators of stomach activity. In further embodiments, sensor 14 includes one or more mechanical sensors to detect motion of stomach 12, distention of stomach 12, or other mechanical indicators of stomach activity. The invention is not limited to mechanical, chemical and electrical sensors, however, but includes other types of sensor as well, such as temperature sensors or auditory sensors.

Physiological parameters sensed by sensor 14 are supplied to IMD 16. IMD 16 measures a characteristic of a physiological parameter sensed by sensor 14. For a sensed physiological parameter, IMD 16 tracks the parameter over time, measuring the rate of change of the parameter, for example, the amplitude of the parameter, the duration of the parameter, the intensity or concentration of the parameter, or other qualities. MD 16 generates a communication to patient 10 as a function of the measurement.

When sensor 14B comprises a mechanical sensor that senses distension of stomach 12, MD 16 measures and records the sensed distension and generates a communication based on the measurement. The communication may include information concerning the timing of the distension, the rate of distension, the magnitude of the distension, and the like.

In FIG. 1, IMD 16 transmits the generated communication to an external module 20. External module 20 receives the generated communication from IMD 16 and presents information to patient 10 as a function of the communication. As shown in FIG. 1, IMD 16 communicates wirelessly with external module 20 via RF telemetry, but the communication may also be transmitted via a wired connection, an optical connection, or a transcutaneous communication link. In some embodiments, patient 10 carries external module 20 on his person. External module 20 presents information to patient 10 as a function of sensed and measured stomach activity. The information may be presented visually, audibly, tactilely, or in any other manner. External module 20 may be a device dedicated to presenting information pertaining to stomach activity, or external device 20 may be a general purpose device such as a pager, cellular telephone, or personal digital assistant (PDA).

For example, an obese patient or a patient who has had stomach surgery, may have a mechanical sensor 14B that senses distension of stomach 12. IMD 16 measures the magnitude of stomach distension via sensor 14A or sensor 14B, and upon measurement of a large magnitude distension, generates a communication and transmits the communication to external module 20. External module 20 may present patient 10 with information about the distension by, for example, sounding an alarm and displaying a message. In response to the message, patient 10 can change his behavior, such as by discontinuing eating until the distension has subsided.

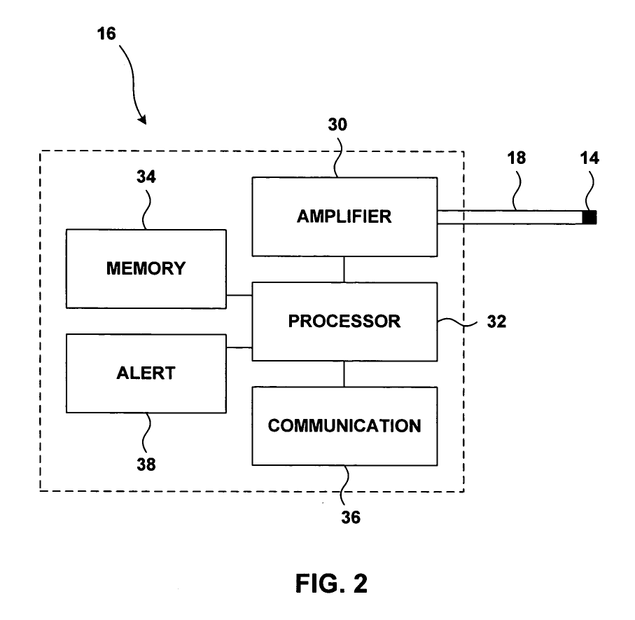

FIG, 2 is a block diagram illustrating an embodiment of the invention. In FIG. 2, IMD 16 is coupled to a sensor 14 by a lead 18. An amplifier 30 receives signals detected by sensor 14. Amplifier 30 amplifies and filters the received signals and supplies the signals to a processor 32. Processor 32 processes the received signals, and analyzes the physiological parameter of interest.

The received signal may be converted to digital values and stored in memory 34. Memory 34 may include any form or volatile memory, non-volatile memory, or both. In addition to data sensed via sensor 14, memory 34 may store records concerning measurements of detected physiological parameters, communications to patient 10 or other information pertaining to operation of IMD 16. Memory 34 may also store information about patient 10. In addition, processor 32 is typically programmable, and programmed instructions reside in memory 34.

Processor 32 determines whether to generate a communication to patient 10 based upon the measurement. As shown below, processor 32 may compare a parameter, or one or more characteristics of a parameter, to a threshold, and may generate a communication when the threshold is surpassed. When processor 32 generates a communication, processor 32 may convey the communication to patient 10 by a number of channels. IMD 16 may include, for example, a communication module 36 to wirelessly transmit the communication to external module 20. In addition to transmitting a communication to an external module 20, communication module 36 may be configured to wirelessly transmit information about the history or status of IMD 16 to the physician for patient 10.

In addition or in the alternative, IMD 16 may include an alert module 38 that is implanted in the body of patient 10. When activated by processor 32, alert module 38 can notify patient 10 directly without an external module. Alert module 38 may, for example, notify patient 10 audibly or by vibration.

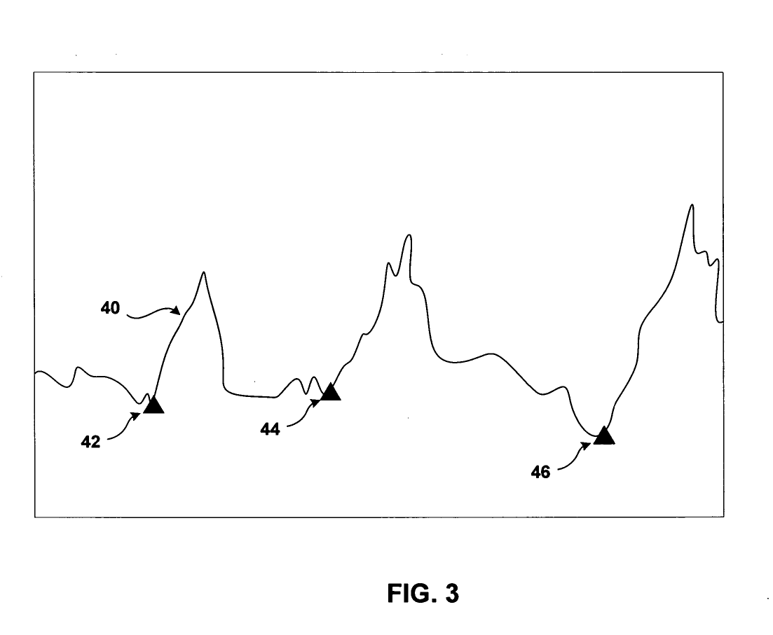

FIG. 3 illustrates analysis of an exemplary physiological parameter. FIG. 3 includes a graphical representation 40 of the blood glucose for patient 10 sensed by sensor 14 over a period of time. Monitoring blood glucose is important for a patient who has been diagnosed with diabetes, and who and treats his condition by regulating his diet and by administering insulin shots. FIG. 3 is demonstrative and does not represent actual measured data. Sensor 14 may sense blood glucose levels chemically, optically, with infrared light, or using any other sensing technique.

Initially, the blood glucose level is stable and at a baseline level. Blood glucose level generally changes with stomach activity, however. In particular, ingestion of a meal typically causes blood glucose levels to rise. After consumption of meals, as indicated by reference numerals 42, 44 and 46, sensor 14 senses a substantial increase in blood glucose. Processor 32 of IMD 16 measures a characteristic of the physiological parameter, such as the amplitude, rate of change, duration of elevated glucose level, or any other characteristic. Further, processor 32 compares the measured characteristic to a threshold value stored in memory 34 and generates a communication to notify patient 10 when the measured characteristic surpasses the threshold. The generated communication can notify patient 10 of his current condition. The communication can further notify patient 10 as to what action patient 10 ought to take to treat his current condition.

The criteria for generating a communication vary from patient to patient. For some patients, a sharp increase in blood glucose may result in the generation of a communication. In other patients, a sharp increase is of less concern than a high amplitude or peak value of the blood glucose concentration. In a further set of patients, the duration of elevated blood glucose may be of special concern. The invention provides for measuring a variety of characteristics of a single physiological parameter.

In addition, processor 32 may measure a characteristic of one physiological parameter as a function of another physiological parameter. There is a relationship, for example, between the blood glucose levels following a meal and the caloric content of the meal. By analysis of blood glucose levels, processor 32 can estimate the caloric intake of patient 10. In an obese patient, an estimate of caloric intake may be of greater interest than blood glucose concentration.

In the event the measured characteristic surpasses the applicable threshold, processor 32 generates a communication to notify patient 10. Patient 10 may respond by, for example, self-administering medication, ceasing eating, or seeking medical attention. IMD 16 continues to monitor the physiological parameter to determine whether the condition is being addressed.

Similar techniques may be applied to physiological parameters other than blood glucose that reflect stomach activity. Accordingly, the invention provides an convenient vehicle for the monitoring and treatment of obesity, diabetes, eating disorders, and the like. In addition, the invention allows the patient to obtain information about his condition and to exercise control over his own health and well-being.

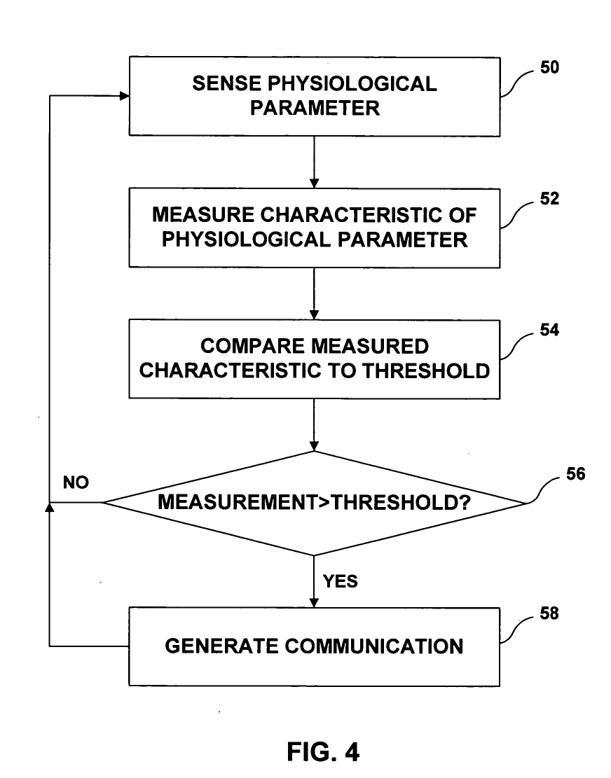

FIG. 4 is a flow diagram illustrating a technique for monitoring one or more physiological parameters that reflect stomach activity. Processor 32 receives data concerning a physiological parameter that reflects stomach activity from sensor 14 (50). Sensor 14 may respond to any of several electrical, mechanical, chemical or other physiological parameters.

Processor 32 processes the data received from sensor 14 and measures one or more characteristics as a function of the sensed physiological parameter (52). The measured characteristic can be a characteristic of the physiological parameter itself, such as the concentration of blood glucose or the magnitude of stomach distension. The measured characteristic can also be a characteristic of a related physiological parameter, such as a measurement of caloric intake as a function of blood glucose levels.

Processor 32 compares the measured characteristic to a threshold value (54) stored in memory 34. When the measured characteristic surpasses the threshold, processor 32 generates a communication that notifies patient 10 of his condition (58). When the measured characteristic does not surpass the threshold, processor 32 may continue to monitor the physiological parameters. In some implementations, a measurement will “surpass” a threshold when the measurement is above the threshold, and in other implementations, the measurement will “surpass” a threshold when the measurement is below the threshold.

The invention further encompasses one or more computer-readable media comprising instructions that cause a processor, such as processor 32, to carry out the techniques of the invention. A computer-readable medium includes, but is not limited to, any magnetic or optical storage medium, ROM or EEPROM.

The preceding specific embodiments are illustrative of the practice of the invention. It is to be understood, therefore, that other expedients known to those skilled in the art or disclosed herein may be employed without departing from the invention or the scope of the claims. For example, the present invention further includes within its scope methods of making and using systems as described herein. Furthermore, the invention includes embodiments that use techniques to sense physiological parameters in addition to those specifically described herein.

Moreover, the invention includes embodiments in which IMD 16 is not be dedicated to sensing stomach activity, but performs other functions as well. IMD 16 may include, for example, an implantable drug delivery system such as any of a number of SynchroMed pumps manufactured by and commercially available from Medtronic Inc. In such embodiments, IMD 16 may actively administer therapy, such as by dispensing insulin or medication, in addition to generating a communication to patient 10.

The invention further includes embodiments in which processor 32 measures a characteristic as a function of two or more physiological parameters. For example, processor 32 may estimate caloric intake as a function of stomach distension, as sensed by a mechanical sensor, and blood glucose levels, as sensed by a chemical sensor.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Many embodiments of the invention have been described. Various modifications may be made without departing from the scope of the claims. These and other embodiments are within the scope of the following claims.

1. A method for monitoring stomach activity of a patient comprising:
sensing a physiological parameter of the patient that changes as a function of activity of a stomach of the patient; and
generating a communication to the patient as a function of the sensed physiological parameter.

2. The method of claim 1, wherein the physiological parameter includes at least one of a blood glucose concentration, an insulin concentration, a body temperature, a distention of the stomach, a stomach acid concentration, a gastric electrical activity and a transabdominal impedance.

3. The method of claim 1, further comprising:
measuring a characteristic of the physiological parameter; and
generating a communication to the patient as a function of the measurement.

4. The method of claim 3, wherein the characteristic of the physiological parameter comprises at least one of a rate of change of the physiological parameter, an amplitude of the physiological parameter, a duration of the physiological parameter, an intensity of the physiological parameter and a concentration of the physiological parameter.

5. The method of claim 3, wherein the characteristic of the physiological parameter is a first characteristic of a first physiological parameter, the method further comprising measuring a second characteristic of a second physiological parameter as a function of the first characteristic.

6. The method of claim 1, wherein generating the communication comprises transmitting a wireless communication to an external module.

7. The method of claim 1, wherein generating the communication comprises activating an implanted alert module.

8. A system comprising:
a sensor to sense a physiological parameter of a patient that changes as a function of activity of a stomach of the patient; and
a processor to generate a communication to the patient as a function of the sensed physiological parameter.

9. The system of claim 8, further comprising a communication module to wirelessly transmit the communication to an external module.

10. The system of claim 8, further comprising an implanted alert module to notify the patient of the communication.

11. The system of claim 8, wherein the sensor comprises a chemical sensor.

12. The system of claim 11, wherein the chemical sensor senses at least one of blood glucose concentration, insulin concentration and stomach acid concentration.

13. The system of claim 8, wherein the sensor comprises a mechanical sensor.

14. The system of claim 13, wherein the mechanical sensor senses at least one of motion of the stomach and distention of the stomach.

15. The system of claim 8, wherein the sensor comprises an electrical sensor.

16. The system of claim 15, wherein the electrical sensor senses at least one gastric electrical activity and transabdominal impedance.

17. The system of claim 8, wherein the sensor comprises a temperature sensor.

18. The system of claim 8, wherein the processor is implantable in the patient.

19. The system of claim 8, wherein the processor is further configured to measure a characteristic of the physiological parameter and to compare the characteristic to a threshold.

20. A system comprising:
sensing means to sense a physiological parameter of a patient that changes as a function of activity of a stomach of the patient;
processing means to generate a communication as a function of the sensed physiological parameter; and
communication means to notify the patient of the communication.

21. The system of claim 20, wherein the processing means is further configured to measure a characteristic of the physiological parameter.

22. The system of claim 21, further comprising a memory means to data associated with the sensed physiological parameter and the measured characteristic.

23. A computer-readable medium comprising instructions that cause a processor to:
sense a physiological parameter of a patient that changes as a function of activity of a stomach of the patient; and
generate a communication to the patient as a function of the sensed physiological parameter.

24. The medium of claim 23, the instructions further causing the processor to:
measure a characteristic of the physiological parameter; and
generate a communication to the patient as a function of the measurement.
